(19) United States
(12) Patent Application Publication
(10) Pub. No.: US 2026/0144510 A1
(43) Pub. Date: May 28, 2026

(54) SYSTEMS AND METHODS FOR MEDICAL IMAGE ACQUISITION THROUGH PRE-SHOT IMAGE ANALYSIS

(71) Applicant: Real Time Tomography, LLC, Villanova, PA (US)

(72) Inventors: Susan Ng, Villanova, PA (US); Peter A. Ringer, Allentown, PA (US); Johnny Kuo, Lancaster, PA (US); Tristan Douglas Maidment, Weehawken, NJ (US); Rodrigo De Barros Vimieiro, Minas Gerais (BR); Lucas Rodrigues Borges, Sao Paulo (BR)

(21) Appl. No.: 19/402,347

(22) Filed: Nov. 26, 2025

Related U.S. Application Data
(60) Provisional application No. 63/726,042, filed on Nov. 27, 2024.

(51) Int. Cl. A61B 6/00; A61B 6/50; A61B 6/58; G06T 7/00

(52) U.S. Cl. CPC A61B 6/488; A61B 6/502; A61B 6/5282; A61B 6/58; G06T 7/0012; G06T 2207/10116; G06T 2207/30068; G06T 2207/30168

(57) ABSTRACT

Systems and methods for analyzing images. The methods comprise using an imaging device to acquire a pre-shot image of a target anatomical area of an individual; analyzing the pre-shot image to assess image quality of one or more regions within the pre-shot image; and determining acquisition parameters for a full-dose image from the one or more measures of image quality. The methods also include acquiring, by the imaging device, a full-dose image using the acquisition parameters determined during analysis of the pre-shot image; and generating a final image from the full-dose image.

### CROSS-REFERENCE TO RELATED APPLICATIONS

The present application claims priority to and the benefit of U.S. Provisional Patent Application Ser. No. 63/726,042 which was filed on Nov. 27, 2024. The content of this U.S. Provisional Patent Application is incorporated herein by reference in its entirety.

### BACKGROUND

### Description of the Related Art

In medical imaging, the ability to detect and characterize anatomic tissue and abnormalities accurately is paramount. This ability is predicated upon obtaining the correct image information during the acquisition process. Historically, phototiming or Automatic Exposure Control (AEC) was used to determine the preferred exposure parameters. This was typically achieved with air-filled ion chambers located in front of the film-screen cassette or the digital detector. Alternative methods included solid-state detectors located behind the film-screen cassette, or thin solid-state detectors bonded or fabricated on the front of the digital detector. In all instances, the size of these phototiming detectors was large compared to the image detector element size or film-screen resolution limits. Often there was either a single AEC detector region, or a very small number of AEC detector regions. The AEC detector region could, in some instances, be moved by the imaging system operator to better accommodate the patient's pathophysiology. Additional information (such as the thickness of the breast, or restrictions on the acquisition technique (e.g., kV/filter combinations or dose limitations) may also be used as input to the AEC.

With the advent of large format digital detectors with fast readout, it has become possible to use the x-ray detector itself to generate a pre-shot image that spans the entire field of view of the detector. These pre-shot images can be acquired at reduced resolution or at the full resolution of the detector, but are typically acquired at a much reduced radiation dose to the patient. Today, the acquisition process typically begins with the acquisition of a pre-shot image, which is obtained using low doses of ionizing radiation before the full-dose standard examination image is acquired. The pre-shot image may be at reduced resolution or at full resolution. While the pre-shot image serves an essential role in informing subsequent imaging decisions, to date the potential for enriching the imaging protocol has not been fully realized.

A significant challenge in optimizing imaging protocols lies in the variability of anatomical features across different patient demographics. The selection of acquisition parameters, including but not limited to the exposure parameters, is not a one-size-fits-all approach. Settings that are ideal for one patient may be suboptimal for another. Current practices primarily focus on estimating the radiographic attenuation of the patient in the pre-shot image. In general, the estimate is made in a fixed square or rectangular region of interest, either in whole or in patches, and does not fully account for the anatomic, demographic, or situational differences that are captured in the pre-shot image.

### SUMMARY

This document concerns implementing systems and methods for analyzing images. The methods comprise: acquiring, by an imaging device, a pre-shot image of a target anatomical area of an individual; analyzing, by a processor, the pre-shot image to determine one or more regions within the pre-shot image to assess image quality, calculate one or more measures of image quality in the one or more regions, and determine acquisition parameters for a full-dose image from the one or more measures of image quality; acquiring, by the imaging device, a full-dose image using the acquisition parameters determined during analysis of the pre-shot image; and generating a final image from the full-dose image.

This document also concerns a system comprising a processor and a non-transitory computer-readable medium that stores instructions that are configured to, when executed by the processor, cause the processor to: obtain a pre-shot image of a target anatomical area of an individual that was acquired by an imaging device; analyze the pre-shot image to determine one or more regions within the pre-shot image to assess image quality, calculate one or more measures of image quality in the one or more regions, and determine acquisition parameters for a full-dose image from the one or more measures of image quality; control an imaging device to acquire a full-dose image using the acquisition parameters determined during analysis of the pre-shot image; and generate a final image from the full-dose image.

### BRIEF DESCRIPTION OF THE DRAWINGS

This disclosure is facilitated by reference to the following drawing figures, in which like numerals represent like items throughout the figures.

FIG. 1 provides an illustration of a system implementing the present solution.

FIG. 2 provides an illustration showing a U-net architecture.

FIG. 3 provides an illustration of a computing device.

FIG. 4 provides a flow diagram of an illustrative method for operating a system in accordance with the present solution.

FIG. 5 provides a flow diagram of an illustrative method for analyzing a pre-shot image in accordance with the present solution.

FIGS. 6A-6D (collectively referred to as “FIG. 6”) provide images showing samples of detected anatomic landmarks of mediolateral oblique view mammography and craniocaudal view mammography.

FIGS. 7A-7C (collectively referred to as “FIG. 7”) provide images showing occlusions detected in pre-shot images.

FIGS. 8A-8B (collectively referred to as “FIG. 8”) provide images showing phantoms detected in pre-shot images.

FIGS. 9A-9B (collectively referred to as “FIG. 9”) provide images showing areas for pixel value estimation in pre-shot images.

FIGS. 10A-10D (collectively referred to as “FIG. 10”) provide images showing cases in which breast positioning problems have been detected using the present solution.

FIG. 11 provides a flow diagram of a method for analyzing and using a pre-shot image to determine the acquisition parameters for a full-dose image.

### DETAILED DESCRIPTION

It will be readily understood that the solution described herein and illustrated in the appended figures could involve a wide variety of different configurations. Thus, the following more detailed description, as represented in the figures, is not intended to limit the scope of the present disclosure but is merely representative of certain implementations in different scenarios. While the various aspects are presented in the drawings, the drawings are not necessarily drawn to scale unless specifically indicated.

The present solution may be embodied in other specific forms without departing from its spirit or essential characteristics. The described embodiments are to be considered in all respects only as illustrative and not restrictive. The scope of the present solution is, therefore, indicated by the appended claims rather than by this detailed description. All changes which come within the meaning and range of equivalency of the claims are to be embraced within their scope.

Reference throughout this specification to features, advantages, or similar language does not imply that all the features and advantages that may be realized should be or are in any single embodiment of the invention. Rather, language referring to the features and advantages is understood to mean that a specific feature, advantage, or characteristic described in connection with an embodiment is included in at least one embodiment of the present invention. Thus, discussions of the features and advantages, and similar language, throughout the specification may, but do not necessarily, refer to the same embodiment.

Although the present solution can be used for a wide range of medical imaging applications (as detailed below), x-ray breast imaging (mammography) will be used primarily to illustrate the applicability of the present solution.

The present document describes improvements in the field of medical imaging, specifically focusing on implementing systems and methods for utilizing information from pre-shot images to acquire superior full-dose medical images. A “pre-shot” (or low-dose) image and a “full-dose” (or standard dose) image are referenced in the following discussion of the present solution. The pre-shot images are x-ray images acquired for the purposes of estimating how to acquire the full-dose image on a given patient. The radiation dose for a pre-shot image is a dose of radiation that is significantly less than the imaging device's typical or usual full-dose (e.g., the average or median full-dose defined over a range of patients having a variety of tissue compositions and body types, or the full-dose required to image an anthropormophic phantom representing a typical patient). For example, a low-dose of radiation may be no more than approximately 5% of the imaging device's typical full-dose, no more than approximately 10% of the imaging device's typical full-dose, or no more than some other threshold that is significantly less than the imaging device's typical full-dose. In some jurisdictions, there are legal limitations placed on the maximum dose for an x-ray pre-shot image, and these limitations are incorporated into this solution. Such limitations are detailed below.

In contrast, a “full-dose image” is a diagnostic image captured using the conventional (or standard) clinical radiation intensity (or an intensity that is within a range of intensities) that the imaging device uses during its main imaging operation for a given patient. The full-dose image for a given patient could be acquired at a lesser or greater radiation dose than the typical full-dose. For example, a half-dose image (an image acquired with approximately half the radiation dose of the typical full-dose image) could satisfy the definition of a full-dose image, if the processing algorithm judges that sufficient image quality will be obtained with a half-dose image for a given patient. In general, the intent of acquiring the full-dose image is to ensure that the image meets the clinical criteria needed for diagnostic purposes for that patient. Thus, it is in general desirable to ensure image quality over dose, provided patient safety guidelines are followed in regard to the full-dose image acquisition. This discussion of dose is intended to ensure that limits to the radiation dose (x-ray), field strength (MRI), heating and mechanical indices (ultrasound), etc. are applied to ensure the safety of the patient, and that the selection of the full-dose is optimized to maximize (or a good approximation thereof) the image quality obtained as a function of dose. Again, in some jurisdictions there are legal limitations placed on the maximum dose for an x-ray image (with similar limitations for other medical imaging devices, e.g., maximum gradient strength in MRI), and these limitations are incorporated into this solution. Such limitations are discussed in more detail below.

The pre-shot image contains a wealth of information that can inform and improve the final full-dose imaging process. The present solution provides a novel method and system that enhances the medical imaging process by exploring and analyzing the rich data located within pre-shot images. Instead of relying solely on conventional methods for determining exposure parameters, the present solution utilizes comprehensive image and data analytics to harness the full potential of the pre-shot image and related acquisition data, ultimately refining a set of full-dose acquisition parameters and directing the acquisition of the full-dose images based on the values of those parameters.

For the purposes of explanation, a detailed description will be given with reference to x-ray imaging of the breast (mammography). In this context, the exposure parameters most commonly in conventional AEC or phototimer systems refer to the factors that influence the amount and characteristics of the radiation reaching an x-ray detector. The exposure parameters may include, for example, a kilovolt (kV) potential difference across an x-ray tube, a milliamperage (mA) current in the x-ray tube at the time of exposure, and an exposure time of radiation(s), but may also include the x-ray target material (e.g., molybdenum, rhodium, or tungsten), and/or filter type (e.g., molybdenum, rhodium, silver, aluminum or copper filters, filter thickness), etc. By comparison, the acquisition parameters presented in this invention include the aforementioned exposure parameters, but can also include additional parameters; examples include, but are not limited to: selection of filter types (e.g., wedge filter(s), bow-tie filters, etc.), detector settings (gain, offset, noise floor, readout speed, etc.), mechanical scanning speed (in systems with a scanning motion), and other parameters as would be understood by one skilled in the art.

The present solution provides systems and methods of a real-time technique to reduce or avoid disruption or delay to the acquisition workflow. The present solution features an enhanced decision-making framework with in-depth information extraction, automatic detection of hardware failure, assessment of patient anatomy and positioning, and tailored acquisition parameter optimization. With regard to in-depth information extraction, the present solution conducts a thorough analysis of the pre-shot image to identify key anatomical features and contextual information that contribute to the improvement of the acquisition parameters. The decision-making framework integrates pre-shot analysis into the imaging workflow, facilitating informed choices regarding acquisition parameters, which improves the overall diagnostic accuracy and effectiveness. The in-depth information extraction comprises a thorough analysis of the pre-shot image to identify key anatomical features and contextual information that combined with other pre-shot data (e.g., breast thickness, breast density, etc.) contribute to the improvement of the determination of the acquisition parameters for the full-dose image. In instances where the pre-shot image is determined to be of insufficient quality, it would be possible via this solution to halt the acquisition process prior to taking the full-dose image. This avoids full-dose irradiation of the patient when suboptimal image acquisition would ultimately require retakes, sparing the patient from unnecessary radiation exposure and dose.

With regard to automatic detection of hardware failure, the present solution analyzes the pre-shot images to detect a variety of hardware failure modes or hardware limitations before the patient is exposed to the full radiation dose. The hardware failures can include, but are not limited to, image detector failure, incorrect collimation, or foreign objects in the beam.

With regard to assessment of patient anatomy and positioning, the present solution can verify the correct positioning of a patient before the patient is exposed to the full radiation dose. This functionality aids technicians in ensuring that the patient alignment is optimal (or at least suitable for a quality image), which can lead to immediate enhancements in image quality.

With regard to tailored acquisition parameter optimization, the present solution gathers insights from the pre-shot images and pre-shot data to customize the imaging protocol to better accommodate the specific anatomical conditions of the patient. For example, assessing the density, shape and other features of a part of the patient's body critically may inform adjustments of the imaging protocols necessary for high-quality imaging. The imaging protocols can include, but are not limited to, information defining (i) patient positioning to correctly align the target anatomical region with the imaging modality, (ii) acquisition parameter setting(s), and/or (iii) image processing technique(s).

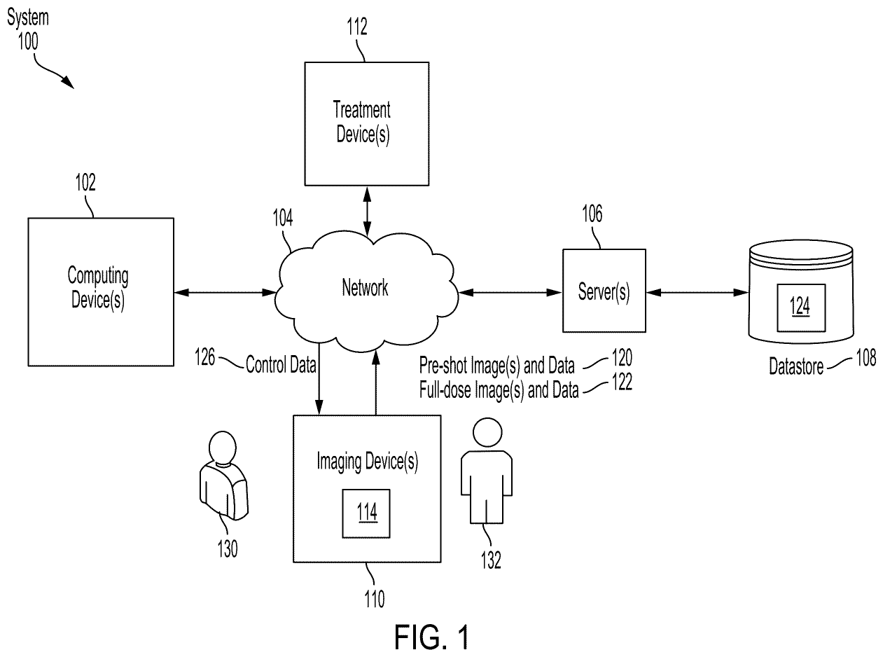

FIG. 1 provides an illustration of a system 100 implementing the present solution. System 100 is generally configured to perform image acquisition, image analysis, image processing or reconstruction, treatment plan generation, and/or treatment plan implementation. In this regard, system 100 comprises computing device(s) 102 that can communicate with other devices via network 104. Network 104 can include, but is not limited to, the Internet and/or an intranet. The other devices include imaging device(s) 110 and treatment device(s) 112. The imaging device(s) 110 is(are) configured to acquire image(s), analyze image(s), process or reconstruct images, and store image(s) in a datastore 108. The image(s) may be additionally or alternatively analyzed by computing device 102. Accordingly, image(s) may be communicated from imaging device(s) 110 to computing device 102 via network 104. This communication can be synchronous or asynchronous.

The image analysis may be performed for various purposes. For example, pre-shot image(s) and pre-shot data 120 may be: (i) acquired and analyzed to obtain acquisition parameters for full-dose image(s) and data 122 that are to be acquired by the imaging device(s) 110; (ii) analyzed to detect and/or characterize anatomic tissue and/or abnormalities for a patient 132; and/or (iii) analyzed to facilitate generation of a treatment plan. The acquisition parameters may be sent from computing device 102 to imaging device(s) 110 as or along with control data 126. The control data 126 can include, but is not limited to, commands for controlling operations of imaging device(s). Any suitable commands for controlling operations of imaging device(s) that are or that may become known to one of skill in the art may be used here.

The treatment plan may be implemented by operator 130 and/or treatment device(s) 112. The treatment device(s) 112 can include, but is(are) not limited to, tissue sampling device(s), radiation application device(s) and/or robotic system(s). The tissue sampling device(s) may be used to perform tissue biopsies. including but not limited to percutaneous biopsies. The radiation application device(s) may be used for treating cancer or other diseases. The robotic system(s) can include, but are not limited to, articulating arms with grippers or other end effector tools (e.g., scalpels, scissors, forceps, suture needles). The robotic system(s) may be used, for example, to facilitate the removal of tissue(s), tumor(s) and/or cyst(s).

After processing the pre-shot images and data 120, and/or after the possible output of recommendations to the operator 130 and receipt of appropriate response(s), the computing device(s) 102 may communicate control data 126 to the imaging device(s) 110 so as to abort or continue the acquisition sequence. Continuance could include modifying and repeating the pre-shot imaging process or proceeding to acquire the full-dose image(s) and data 122. If a full-dose image is acquired, both the full-dose image and full-dose data 122 may be communicated to the computing device 102 for post-processing. The post-processing steps can include, but are not limited to, verification of the anatomic landmark detection, occlusion identification, confidence estimation, pixel statistics, morphology analysis, and/or positioning measurements. Additionally, the image(s) may be sent for image processing and/or image reconstructions in accordance with any suitable techniques. Examples of such known image processing and image reconstruction techniques are those described in U.S. Non-Provisional Patent Application Publication No. 2023/0177686 and U.S. Pat. No. 8,233,690, the disclosures of which are fully incorporated into this document by reference.

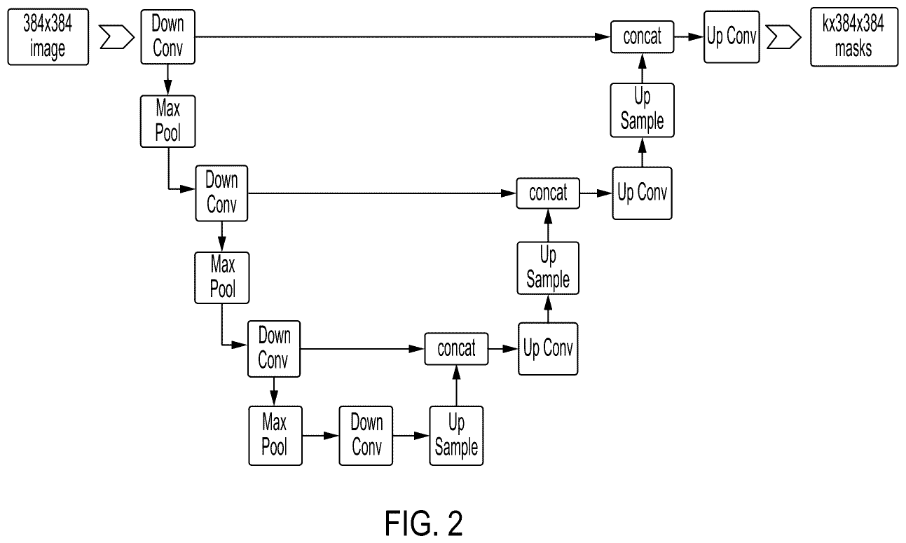

The present solution relies on the correct identification of landmarks and the correct selection of regions of interest (ROIs). To achieve those, computer algorithms may be applied, including various artificial intelligence (AI) algorithms. AI algorithms can include, but are not limited to, convolutional neural networks (e.g., U-Nets), deep neural networks (e.g., residual networks (ResNets)), generative adversarial networks (GANs), and/or supervised machine learning algorithms (e.g., support vector machines (SVMs), decision trees, and/or random forests). Additionally or alternatively, other methods can be used such as histogram analysis, principal component analysis (PCA), and/or histogram of oriented gradients (HoG). FIG. 2 shows an illustrative U-net architecture that can be implemented by system 100 due to its flexibility, small memory footprint and low computational cost.

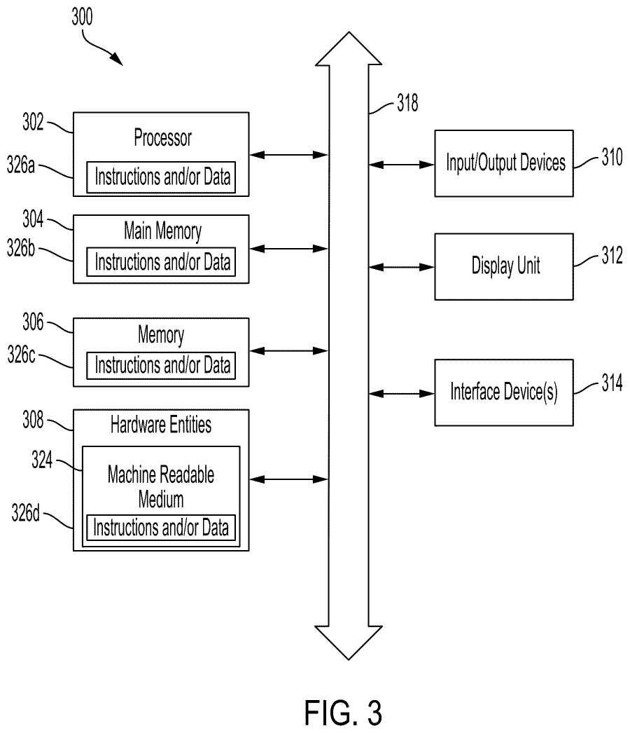

FIG. 3 shows an illustrative architecture for a computing device 300. Computing device(s) 102 of FIG. 1, server(s) 106 of FIG. 1, imaging device(s) 110 of FIG. 1 and/or treatment device(s) 112 of FIG. 1 is/are the same as or similar to computing device 300. As such, the discussion of computing device 300 is sufficient for understanding the listed components 102, 106, 110, 112 of FIG. 1.

Computing device 300 may include more or less components than those shown in FIG. 3. However, the components shown are sufficient to disclose an illustrative example implementing the present solution. The hardware architecture of FIG. 3 represents one implementation of a representative computing device configured to receive information, process the received information, transmit information and/or control operations of one or more external device, as described herein. As such, the computing device 300 of FIG. 3 implements at least a portion of the method(s) described herein.

Some or all components of the computing device 300 can be implemented as hardware, software and/or a combination of hardware and software. The hardware includes, but is not limited to, one or more electronic circuits. The electronic circuits can include, but are not limited to, passive components (e.g., resistors and capacitors) and/or active components (e.g., amplifiers, memory chips, digital or analog interfaces, and/or microprocessors). The passive and/or active components can be adapted to, arranged to and/or programmed to perform one or more of the methodologies, procedures, or functions described herein.

Computing device 300 comprises, a processor 302, main memory 304, memory 306, hardware entities 308, input/output device(s) 310, a display unit 312, and interface device(s) 314 that are connected to and accessible by other portions of computing device 300 through system bus 318. The input/output device(s) 310 may be referred to as a user interface configured to facilitate user-software interactions for controlling operations of the computing device 300. The input devices may include, but are not limited to, a physical and/or touch keyboard, switches or switch boxes, computer mice, track pads, track balls, and other computer-human interface devices. The input devices can be connected to the computing device 300 via a wired or wireless connection (e.g., a Bluetooth® connection). The output devices can include, but are not limited to, a speaker and/or light emitting diodes. Display unit 312 may also be provided as an output device of the user interface. Display unit 312 can include, but is not limited to, a computer monitor, liquid crystal display (LCD), and/or printer(s). The listed display devices may be in color or in grey-scale. Interface device(s) 314 may provide a system interface configured to facilitate wired or wireless communications to and from external devices (e.g., other computing devices such as imaging devices, treatment devices, servers, and network nodes such as routers, networks switches and network access points).

At least some of the hardware entities 308 perform actions involving access to and use of main memory 304 and memory 306, which can be a random access memory (RAM), a disk drive, flash memory, a universal serial bus (USB) drive and/or another hardware device that is capable of storing instructions and data 326a . . . 326d (which this disclosure may refer to collectively as 326). The instructions and data 326 can also reside, completely or at least partially, within the main memory 304, memory 306, and/or within the processor 302 during execution thereof by the computing device 300. Each memory described will be or include one or more non-transitory computer-readable media. Hardware entities 308 can include a disk drive unit comprising a machine-readable storage medium 324 on which is stored one or more sets of instructions and data 326 (e.g., software code) configured to implement one or more of the methodologies, procedures, or functions described herein. The term machine-readable media, as used here, refers to a single medium or multiple media (e.g., a centralized or distributed database, and/or associated caches and servers) that store one or more sets of instructions and data 326. The term machine-readable media also refers to any medium that is capable of storing, encoding or carrying a set of instructions for execution by the computing device 300 and that cause the computing device 300 to perform any one or more of the methodologies of the present disclosure.

The pre-shot image and data 120 of FIG. 1 may be processed by processor 302. Processor 302 can include, but is not limited to, a central processing unit (CPU), a graphics processing unit (GPU), a field programmable gate array (FPGA), a tensor processing device (TPU), ARM chip(s), and/or other processing devices. The processor 302 may use a combination of memory 304, 306 and/or external memory (e.g., datastore 108 of FIG. 1) to store the pre-shot image(s) and data 120, trained machine learning algorithms, training algorithms for the machine learning algorithms, training data, and/or other data, algorithms and code as needed to process these data.

In scenarios where time is a scarce resource, such as in many medical imaging systems, GPU-based systems are particularly well-suited for implementation. The massively parallel architecture of GPUs enables efficient processing of large datasets, making them an ideal choice for applications requiring rapid data processing. As stated above, TPUs and other architectures designed to accelerate software parallelization may also be appropriate choices for processing the images and data. Alternatively, FPGAs are commonly integrated into medical imaging detectors and offer a compact and power-efficient solution for real-time data processing. Their programmability allows for flexibility in adapting the methods and systems of this disclosure to various application-specific requirements.

To date, implementations of the present solution have run on a computing device in 50-100 ms. The constraints on the execution of the processing time are dictated by the detector in question. The time between the pre-shot image and the full-dose standard image can be as short as a few 10's of milliseconds, and as long as several seconds. It is most common that the time between the pre-shot image and the full-dose images is between 1 and 2 seconds. Therefore, a goal of the present solution is to keep processing time under one second. In some implementations, it may be necessary to keep the processing time under 500 ms, 200 ms or even 30 ms. This scenario may be referred to as real-time processing, as the processing occurs in the natural time window between the pre-shot and full-dose imaging.

Stated another way, the systems and processes as described herein may be implemented on a CPU, GPU, FPGA or any other graphical or computational device, including the detector itself. The systems and processes may be provided along with a standard image reconstruction tool such as an imaging system configured to perform digitally reconstructed radiographs (DRR), or as a separate set of tools and additional options that may be incorporated into an imaging system via a series of customer purchases or upgrades. Similarly, a portion of the systems and processes as described herein may be provided with an imaging system, while the remaining systems and processes may be incorporated into an imaging system via a series of customer purchases or upgrades.

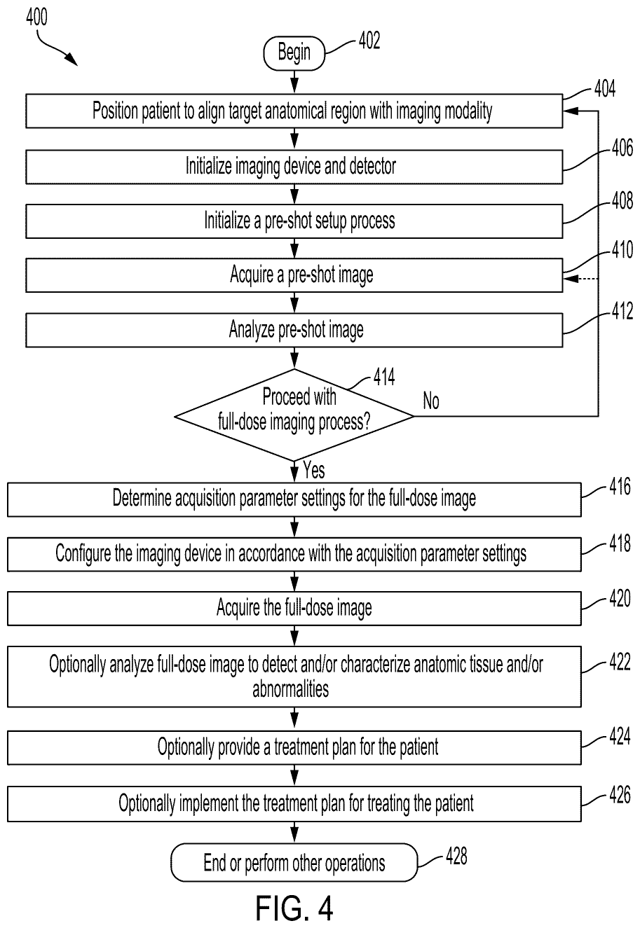

FIG. 4 provides a flow diagram of an illustrative method 400 for operating a system (e.g., system 100 of FIG. 1). Method 400 may include more or less operations than that shown. The operations of method 400 may be performed in the same or different order than that shown. Method 400 is described in a digital mammography context; the present solution is not limited in this regard. Method 400 may be performed by computing device 102 of FIG. 1, server(s) 106 of FIG. 1, imaging device(s) 110 of FIG. 1, computing device 300 of FIG. 3, and/or a processor 302 of FIG. 3. Method 400 is described below in relation to a system. The system can include one or more of these listed device(s).

Method 400 begins in block 402. In block 404, a patient is positioned so as to align a target anatomical region with an imaging modality, including setting the position and/or orientation of the imaging device. The imaging modality can include, but is not limited to, radiography (x-ray imaging), fluoroscopy, mammography, computed tomography (CT), magnetic resonance imaging (MRI), ultrasound imaging, optical imaging, optical tomography, x-ray phase contrast imaging, x-ray dark-field imaging, etc. The target anatomical region can include, but is not limited to, a breast, thorax, abdomen, head, pelvis, hand, foot, extremities, etc. A radiology technologist (e.g., user 130 of FIG. 1) may position the patient and compress the patient's breast accordingly. Aspects of the acquisition such as compression force, tissue coverage, nipple position, and others can affect the quality of the examination and may limit the usability of the images. Thus, it is important that positioning of the patient be done well. Note that certain of these data are directly measurable by the acquisition (such as compressed breast thickness and compression force applied), and these can serve as input data (e.g., pre-shot data 120) to an algorithm implemented by a computing device (e.g., computing device(s) 102, imaging device 110, and/or server(s) 106 of FIG. 1).

Next in block 406, a detector (e.g., detector 114 of FIG. 1) of an imaging device (e.g., x-ray imaging device 110 of FIG. 1) is initialized. The detector can include, but is not limited to, a digital x-ray detector. This initialization operation may involve setting the detector to a pre-shot context (i.e., to capture a pre-shot image rather than a full-dose image) such as setting the appropriate detector gain, image resolution, etc. The operations of block 406 may also involve setting the appropriate parameter(s) for a particular hardware configuration of the imaging device including using pre-shot acquisition parameters such as tube voltage, tube current, exposure time (seconds), and focus spot size.

Next, the hardware and/or software of a pre-shot analysis process may optionally be initialized in block 408. Depending on the platform in which the method was implemented (e.g., GPU vs CPU vs FPGA), initialization of the analysis process may be required as some hardware and/or software may have to be “warmed up” to avoid extra processing time during the first time the analysis is performed. The pre-shot analysis process may be initialized on the imaging device 110 and/or the computing device 102 or 106. This can be done before, after, or simultaneous with the operations of block(s) 404 and/or 406 depending on the time required to initialize that analysis process. For example, in GPU computing the first time an algorithm is executed requires additional time to pre-compile shaders and initialize drivers before the actual task can start. Thus, it is common to perform this extra step (in block 408) prior to acquiring or analyzing the pre-shot image (in blocks 410 and 412) to ensure that the data analysis (in block 412) meets the timing requirements. This step helps ensure the actual image analysis is performed in real-time.

Block 410 involves performing operations by the imaging device to acquire a low-dose pre-shot image. The imaging device will acquire the low-dose pre-shot image by using a x-ray tube voltage (kV), x-ray filter, and/or x-ray target that may be different from the full-dose image, reducing the current delivered to the x-ray tube (mA), reducing the total exposure time(s) of the imaging operation or reducing the product of the mA and exposure time to reduce radiation intensity. Since the pre-shot image is captured using a relatively low radiation dose, the image may have a relatively low image quality as compared to a diagnostic image. In some embodiments, the resolution of the resulting pre-shot image may be smaller than the resolution of an image obtained during a full-dose acquisition process to improve per pixel noise. Alternatively, some systems may use an image resolution equal to the full detector resolution size in the pre-shot image capture process.

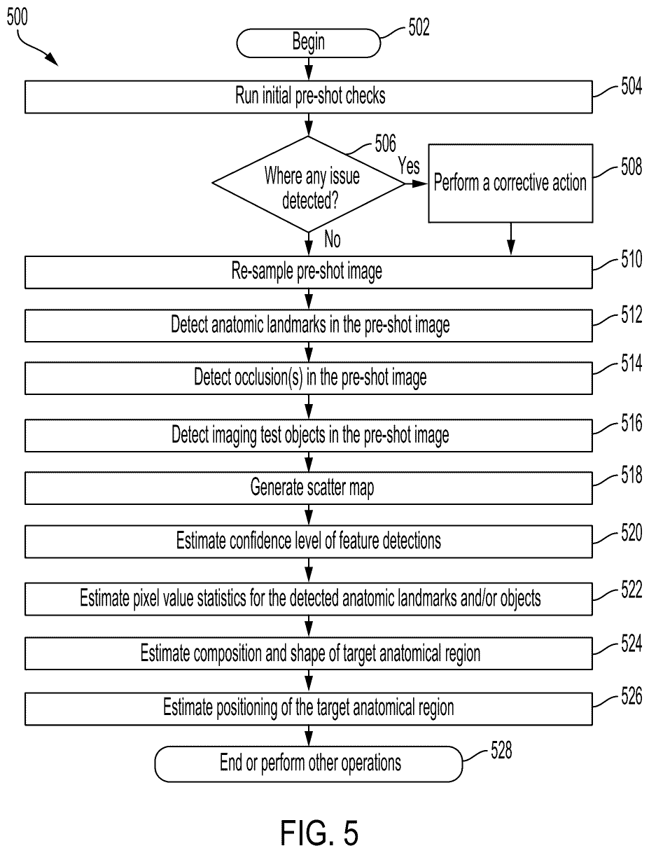

The pre-shot image is analyzed in block 412. The pre-shot image analysis may be called by the acquisition application. This process may take a pre-shot image as an input, and optionally a configuration file that defines the configuration of the particular equipment setup (e.g., detector, x-ray source, etc.) that were used during the pre-shot acquisition process. The pre-shot image analysis measures pre-shot image quality in a number of ways, as detailed in method 500 of FIG. 5. For example, pixel values in different regions of the pre-shot image can be extracted and used quantitatively to assess image quality, target anatomical region positioning (e.g., breast positioning) can be evaluated, target anatomical region shape (e.g., breast shape) can be measured, target anatomical region composition (e.g., breast composition) can be measured, the detection of other objects in the field can be assessed e.g., implants (e.g. breast implants), medical device(s) (e.g., pacemakers), tool(s) (e.g., breast compression paddles), medical instrument(s) (e.g., biopsy devices), and/or other features.

The measure of image quality may be a function of whether or not the target anatomical region is properly oriented, whether or not the image(s) allows masks to be generated for particular regions of interest in the image, and/or whether there is more than a threshold signal-to-noise ratio (SNR) value in an x-ray image. The SNR may be determined by, for example, estimating noise in an x-ray image by (i) distinguishing between areas of anatomy and areas that do not include anatomy, (ii) calculating a ratio between the area/volume of the anatomy and the area/volume of the non-anatomy and/or (iii) comparing the calculated ratio to a threshold value The present solution is not limited to SNR thresholding, since other metrics may additionally or alternatively be considered. Example methods of measuring image quality are described in U.S. Pat. No. 12,250,738, the disclosure of which is fully incorporated into this document by reference.

Method 400 continues with a decision block 414 where a decision is made as to whether or not to proceed with a full-dose imaging process. Results from the pre-shot analysis are used to decide whether to (i) proceed with acquisition of a full-dose image [414:YES] or (ii) abort the image acquisition [414:NO] and provide feedback to the technician on how to improve the full-dose acquisition. For example, the system may determine to proceed with a full-dose image acquisition when results of the pre-shot image analysis indicate that the pre-shot image exhibits at least a threshold measure of image quality and when the breast is properly positioned and adequate tissue coverage is visualized in the image. The system may determine to abort the full-dose image acquisition process when results of the pre-shot image analysis indicate that the measured image quality is less than a threshold, or the breast positioning or breast coverage is inadequate. The system may provide feedback to the technician when the pre-shot image analysis indicates the image quality is less than a threshold image quality. Such feedback may include providing feedback, such as changing one or more aspects of the hardware configuration of the imaging device, or by changing the position or orientation of the target anatomical region.

The decision process will be informed by the measures detailed in FIG. 5 and the related description. Each of the factors assessed in method 500 could influence the decision, including the results of the initial pre-shot check, the anatomic landmarks detected (or not detected), the absence (or presence) of occlusions, the determination that a test object is present, together with the assessment of confidence of these factors. The decision method is based on a series of rules and heuristics, such as a decision tree, an AI algorithm, an deep-learning algorithm or a rule-based algorithm.

An example of the decision process is presented here. If a blank pre-shot image was returned by the detector, then acquisition would be aborted. The pre-shot image and the pre-shot data can be used to predict the acquisition parameters needed for the full-dose image 416. If these parameters fall outside the normal operating range for the imaging device and detector, then the image would be aborted. If the breast were not completely imaged, then the operator 130 would have the option to abort the image. If there was an occlusion determined to be the patient's nose or glasses or forehead then the image would be aborted (assuming the imaging task is mammography). The full algorithm considers the myriad factors that ensure an optimal image is acquired.

Depending on the decided course of action, the pre-shot image acquisition process could be repeated immediately. This is shown by scenario [414:NO] in which method 400 returns to block 410. Alternatively, the technologist may be required to reposition the patient or otherwise improve the conditions for acquisition before a new pre-shot image can be acquired. This is shown by scenario [414:NO] in which method 400 returns to block 404.

Otherwise [414:YES], method 400 continues to block 416 where full-dose image acquisition parameter settings and/or device configuration settings are determined for acquisition of the full-dose image(s). In some scenarios, the acquisition parameters are pre-defined for full-dose images. In other scenarios, the operations of block 416 can involve: obtaining information provided by the pre-shot image analysis; and using the obtained information to estimate the acquisition parameters, the patient dose, and/or other device configurations.

In the absence of defects in the image (e.g., occlusion or poor positioning) that would result in the acquisition process being aborted, then the acquisition parameters for the full-dose acquisition will be estimated. The estimation will be based on the signal identified within the detected anatomic landmarks 512. For example, if the pre-shot image analysis results in an indication that the tissue density is greater than a threshold amount, the x-ray tube voltage (KV) parameter may be increased as compared to the pre-defined value for that parameter. The present solution is not limited to the particulars of this example.

When estimating the acquisition parameters, it is important to estimate the radiation dose to the organ(s) of interest in the field of irradiation. This information may include pixel value statistics, target anatomical region composition, target anatomical region shape, and other information. The quantitative dosimetry values can include, but are not limited to, dose, dose rate, maximum dose, minimum dose, mean dose, volume, tissue-phantom ratio, conformity index, dose-volume histogram, and/or S-value.

As noted above, the full-dose acquisition parameters can include, but are not limited to, a kilovolt (kV) voltage across an x-ray tube, a milliamperage (mA) current in the x-ray tube at the time of exposure, an exposure time of radiation, the x-ray target material (e.g., molybdenum, rhodium, or tungsten), and/or filter type (e.g., aluminum filter or copper filter or wedge filter) and filter thickness. Note that additional information, such as the operating limitations of the x-ray tube and generator (e.g., minimum and maximum values of the kV, mA, time, and limitations to the power (related to the product of the kV and mAs), the instantaneous and long-term heating of the x-ray anode and x-ray tube/housing, etc.), dose restrictions of the patient examinations, and guidelines for common usage (such as the breast phantom dose enumerated in the US MQSA or the “acceptable” and “achievable” clinical doses from the European guidelines for quality assurance in breast cancer screening and diagnosis, Fourth Edition) may also be considered in block 416. For example, the full-dose of radiation could be compared against achievable and acceptable standards—that is a radiation dose that should be achievable for a particular medical exam, or an acceptable dose that is higher than the achievable dose, but is still considered acceptable or safe—and restrictions on the acquisition parameters for the full-dose image might be imposed. Similarly, there may be absolute limits imposed by local or national laws on one or more of the acquisition parameters (e.g., maximum mAs) or the patient dose (e.g., maximum dose for a breast of a given thickness, or the maximum dose to a particular test object or phantom).

Block 418 involves configuring the imaging device in accordance with the acquisition parameter settings determined in block 416. The full-dose image is then acquired in block 420 using the acquisition parameter settings determined in block 416.

The full-dose image is optionally analyzed in block 422 to detect and/or characterize anatomic tissue and/or abnormalities. Example techniques for analyzing images to detect anatomic tissue and/or abnormalities include, without limitation, U.S. Pat. No. 12,450,738, the disclosure of which is incorporated into this document by reference.

A treatment plan may be provided in block 424 for the patient (e.g., individual 132 of FIG. 1). For example, the treatment plan may be to perform a biopsy. Any technique for generating and/or providing a treatment plan based on image analysis that is known, or that becomes known, to those of skill in the art, can be used here. The treatment plan may be generated using final (i.e., for-presentation to a medical professional) images reconstructed or otherwise from the full-dose image and/or additional full-dose images. Any suitable technique for generating a final image from one or more full-dose images, such as those that reconstruct an image by converting 2D projection data into a 3D image can be used here, such as the processes described in U.S. Pat. No. 12,361,608, the disclosure of which is fully incorporated into this document by reference.

The treatment plan may be implemented in block 426 for treating the patient. Any technique for implementing a treatment plan that is known or that may become known to those of skill in the art can be used here. Examples of such a technique is stereoscopic and tomosynthesis-guided percutaneous biopsy of the breast. Such techniques are image-guided, and the methods presented (400 and 500) are compatible with such image-guided biopsies.

Subsequently, method 400 continues to block 428 where it ends or other operations are performed (e.g., return to 402).

FIG. 5 provides a flow diagram of an illustrative method 500 for analyzing a pre-shot image in accordance with the present solution. Method 500 may be performed in block 412 of FIG. 4 to analyze the pre-shot image, and the results of the analysis may be used in block 416 of FIG. 4 to determine the acquisition parameter for the full-dose acquisition. Method 500 may include more or less operations than that shown. The operations of method 500 may be performed in the same or different order than that shown. Method 500 is described in a digital mammography context; the present solution is not limited in this regard. Method 500 may be performed by computing device 102 of FIG. 1, server(s) 106 of FIG. 1, imaging device(s) 110 of FIG. 1, computing device 300 of FIG. 3, and/or a processor 302 of FIG. 3. Method 500 is described below in relation to a system. The system can include one or more of these listed device(s).

Method 500 begins at block 502. In block 504, the system (e.g., system 100 of FIG. 1) runs initial pre-shot checks. This may involve running checks on the input pre-shot image. These validation, verification and compliance checks include, but are not limited to, a uniformity check, a zero-value detection check, a saturation detection check, and/or a minimal image resolution check.

The uniformity check may involve checking if the pre-shot image has any meaningful information and is not an image containing a single pixel value or a very small range of pixel values. This can indicate errors in the pre-shot image acquisition or corrupted data. The pre-shot image may be considered to include meaningful information when anatomy is detected therein and/or pixel values are not substantially uniform throughout the image. If anatomy is not detected in the image, then the system may conclude that the pre-shot technique caused a relatively poor quality pre-shot image. If the pixel values are uniform through the image, then the system may conclude that the imaging equipment may be experiencing a fault or malfunction. The presence or absence of anatomy in the image may be effected by an AI algorithm, and ML algorithm, a procedural algorithm containing a series of measurement and tests, and other methods now or hereafter known to one skilled in the art.

The zero-value detection check may involve checking if one or more regions of the image contains pixels with a value of zero. This could indicate that the detector did not receive any radiation during the pre-shot acquisition, the detector operated in error, or the image data was corrupted. The zero pixel value may account for noise tolerances. The zero pixel value may be zero, or it may be equal to a non-zero offset value that represents the minimum pixel value of the system. If a threshold number of pixels have zero values, then the system may conclude that the image has errors, corrupted data and/or an insufficient amount of data.

The saturation detection check may involve detecting saturated pixels in the image. Saturated pixels can affect image quality and accuracy of the pre-shot image analysis. The saturated pixels can indicate errors, corrupted data or excessive x-rays. The term “saturated pixel”, as used here, refers to a pixel that has reached its maximum value. This means the pixel is displaying the highest possible value, and that any further increase in signal intensity may not be registered. This could lead to a loss of detail in bright areas.

The minimal image resolution check may involve detecting if the input pre-shot image fulfills a minimum resolution requirement. For example, the system may use 384×384 pixels. Other image sizes can be supported including images as small as 16×pixels and as large as the full-resolution, full-field image produced by the detector. The image size may determine the breadth and depth of the feature analysis that is performed.

The above-described validation, verification and compliance checks ensure that the input pre-shot image is valid and can be processed correctly. If any issues are detected [506:YES], the system can alert the user 130 to take corrective action at this or at later stages as shown by block 508. Corrective actions can include, but are not limited to, aborting a full-dose image acquisition process, modifying the pre-shot acquisition techniques and repeating the pre-shot image acquisition process with similar or altered pre-shot parameters, informing the technologist who would make the decision as to whether or not to proceed with the full-dose image acquisition process, and/or continuing with the full-dose image acquisition process.

Next in block 510, the system performs operations to re-sample the resolution of the pre-shot image. This may involve resolution adjustments to match the pre-shot image to the requirements of the pre-shot analysis method through interpolation techniques. Any suitable interpolation technique can be used here, including but not limited to decimation, linear, bilinear, and/or bicubic interpolation. Re-sampling the image resolution is an important step that enables noise suppression and saves computational resources later in the pipeline, playing an important role in the meeting of memory and speed requirements.

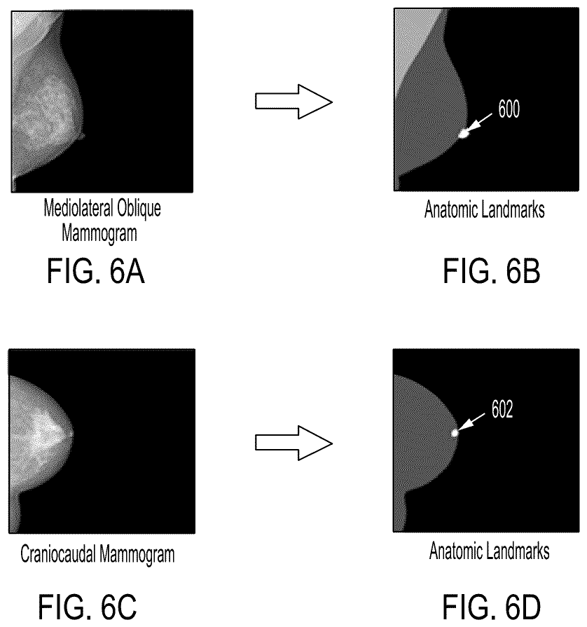

Block 512 involves detecting anatomic landmarks in the pre-shot image. This may involve, for example, nipple detection, pectoralis muscle detection, and/or parenchyma detection. The nipple detection, pectoralis muscle detection and/or parenchyma detection may be achieved using machine learning algorithms, edge detection techniques, texture analysis techniques, and/or histogram-based techniques. The algorithms and techniques can include, for example, those described in U.S. Pat. No. 12,450,738, the disclosure of which is fully incorporated into this document by reference. Other object detection techniques also meet the requirements of this step. Detecting anatomic landmarks can provide valuable information about the patient's anatomy and help to optimize full-dose acquisition parameters. Such information is also important for the determination of patient positioning. FIGS. 6A-6D show samples of detected anatomic landmarks 600, 602 of a mediolateral oblique (MLO) view mammography and a craniocaudal (CC) view mammography. The anatomic landmarks may be marked by, for example: modifying pixel values and/or brightness values to show the anatomic landmarks(s) is a particular color (e.g., red) and/or brightness level; or overlaying a colored mask.

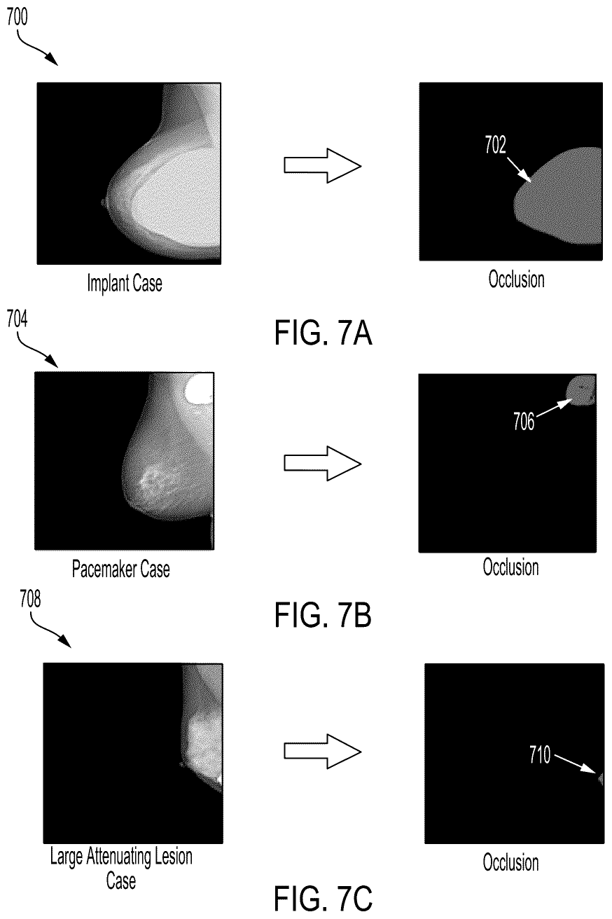

In block 514, the system analyzes the pre-shot image to detect any occlusion in or on the image. An occlusion is an object that appears in the image but which is not part of the target anatomical region. An occlusion can include, but is not limited to, a non-biological object, a foreign object, and/or a body part that is not part of the anatomical region of interest (such as an arm, nose, or chin instead of a breast). The occlusion may be (i) related to the patient's anatomy, medical history and/or medical care, or (ii) related to the pre-shot image acquisition method or (iii) related to the image system or related medical equipment. In this regard, the occlusions can include, but are not limited to, implants, pacemakers, metal, spot paddles, and/or biopsy apparatus. The occlusion may bias the statistical analysis and the resulting calculation of image quality, and consequently adversely affect acquisition parameter settings of the full-dose image. The algorithms and techniques can include, for example, those described in U.S. Pat. No. 12,450,738, the disclosure of which is fully incorporated into this document by reference. FIGS. 7A-7C show occlusions 702, 706, 710 detected in pre-shot images 700, 704, 708. The occlusion may be marked by, for example: modifying pixel values and/or brightness values to show the occlusion(s) is a particular color (e.g., green) and/or brightness level; or overlaying a colored mask thereover. Any suitable object detection technique can be used here. For example, an occlusion may be detected by using machine learning algorithms, edge detection techniques, texture analysis techniques, and/or histogram-based techniques. The present solution is not limited in this regard.

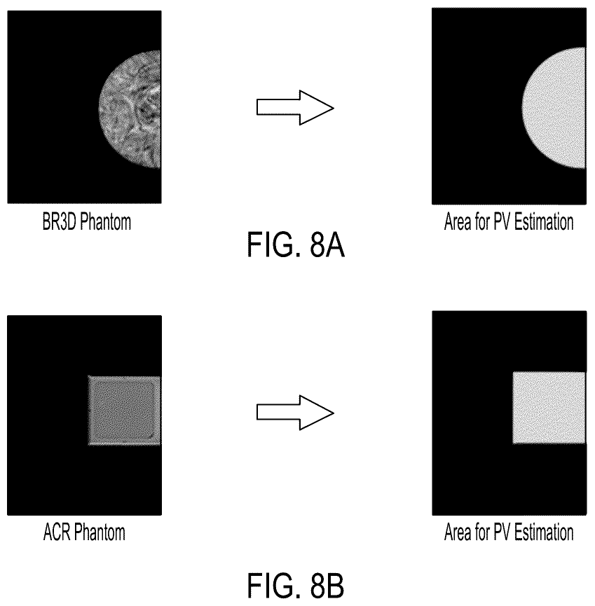

In block 516, the system analyzes the pre-shot image to detect imaging test objects such as, but not limited to, imaging phantoms. Anthropomorphic phantoms are objects that, when imaged, mimic the attenuation, shape and content of human breasts. One example of an anthropomorphic phantom is the BR3D breast imaging phantom (Sun Nuclear, Mirion Technologies) shown in the left image of FIG. 8A. Other phantoms might have shapes and content that are quite different from human breasts. The algorithms and techniques can include, for example, those described in U.S. Pat. No. 12,450,738, the disclosure of which is fully incorporated into this document by reference. One example of this kind of phantom is the American College of Radiology (ACR) breast accreditation phantom shown in left image of FIG. 8B. The imaging test object(s) may be marked by, for example: modifying pixel color values and/or brightness values to show the image test object(s) is a particular color (e.g., blue) and/or brightness level; or overlaying a colored mask thereover. Any suitable object detection technique can be used here. For example, an image test object may be detected by using machine learning algorithms, edge detection techniques, texture analysis techniques, and/or histogram-based techniques. The present solution is not limited in this regard.

In block 518, the system performs operations to estimate or compute a patient-specific scatter map for objects in the pre-shot image. The scatter map represents how x-rays are scattered by different tissues within the target anatomical region of the patient. The scatter map may provide insights about the composition of the tissue being imaged. For example, an imaged breast may comprise different types of breast tissue such as adipose, fibroglandular and/or cancerous tissue. The different types of breast tissue result in unique scatter patterns. This estimate or computation may take into account other attenuating objects, such as metal. The inclusion of scattered radiation in the pre-shot image can lead to incorrect estimates of tissue thickness, density and composition. As an optional step, the pre-shot image may be corrected to remove the influence of the scattered radiation. The modeling of scatter may include, but is not limited to, neural network models (e.g., feedforward, convolutional, recurrent, transformer-based), or other generative modeling techniques known to one skilled in the art.

In block 520, the system performs operations to estimate a confidence level of detected features in the pre-shot image. In the machine learning scenarios, machine learning algorithms may be trained or otherwise configured to compute or otherwise determine confidence levels or values for object detections made in blocks 512, 514 and/or 516. The confidence levels or values can include, but are not limited to, numerical values between zero and one. The confidence levels and/or values may be reported to support the decision making and/or help to improve the accuracy and reliability of the pre-shot image analysis.

In block 522, the system estimates pixel value (PV) statistics for the detected anatomic landmarks, occlusions, and/or imaging test objects. The PV statistics provide quantitative information about the pixels within the detected object. Estimates of PV statistics may involve, for example, a mean estimation, a histogram percentile estimation, and/or a signal-to-noise ratio (SNR) estimation. The mean estimation may be used, for example, to obtain estimations of the mean pixel value of different landmarks detected in the pre-shot image. The histogram percentile estimation may be used, for example, to obtain estimations of the histogram percentiles of different landmarks detected in the pre-shot image. The SNR estimation may be used, for example, to obtain estimations of the SNR of the pre-shot image as detailed above. More commonly, however, these measures and metrics will be combined to make determinations of: the overall image quality; and the image quality of the various anatomic landmarks, occlusions and test objects; the origin or nature of the various anatomic landmarks, occlusions and test objects.

Calculations of the PV statistics can include measures such as, but not limited to, average pixel value, minimum pixel value, maximum pixel value, standard deviation, and/or pixel count. The average pixel value provides insights into the composition and thickness of an object. The minimum and maximum pixel values may allow identification of the most attenuating part and least attenuating part of the object. The standard deviation may provide insights into the texture of the object, the type of the object, and/or the state of the object. The pixel count provides insights into the size of the object. Calculations of the PV statistics can also include, but are not limited to: local intensity peak, global intensity peak, a mean pixel value, variance, skewness, (excess) kurtosis, median pixel value, 10th percentile pixel value, 90th percentile pixel value, interquartile range (IQR) of pixel values, a range of pixel values, mean absolute deviation, robust mean absolute deviation, median absolute deviation, coefficient of variation, quartile coefficient of dispersion, energy level of radiation, and/or root mean square.

Histograms of PV can also be computed, and statistical measures of the histograms can be used to calculate PV statistics and also, for example, to estimate image quality. Statistical measures of the histograms may include, but are not limited to: Mean, Variance, Skewness, (Excess) kurtosis, Median, Minimum, 10th percentile, 90th percentile, Maximum, Mode, Interquartile range, Range, Mean absolute deviation, Robust mean absolute deviation, Median absolute deviation, Coefficient of variation, Quartile coefficient of dispersion, Entropy, Uniformity, Maximum histogram gradient, Maximum histogram gradient intensity, Minimum histogram gradient, and/or Minimum histogram gradient intensity.

Additional PV statistics can be used to detect texture and other image-based features through the use of such measures as run-length features and measures associated with run-length features, neighboring grey level features and measures associated with neighboring grey level features.

All of the statistical measures listed above (paragraphs [0080]-[0082]) generally fall under the description of hand-crafted radiomics features. These features can be used to estimate the PV statistics for the various anatomic landmarks and/or objects. For example, sets of pre-shot and full-dose images can be collected, and divided into training, testing, and validation datasets (for example with a numerical ratio of 80%: 10%: 10%; although other ratios are equally valid). The PV statistics (such as an estimate of a composition and thickness, or a signal-to-noise ratio) of an anatomic landmark(s) or object(s) can then be determined through a process of training a classifier, testing the classifier, and validating the classifier. The classifier may include, but is not limited to, one or more of the following: linear models (e.g., logistic regression), tree-based models (e.g., decision trees, random forests), kernel-based models (e.g., support vector machines), probabilistic models (e.g., naïve Bayes), instance-based models (e.g., k-nearest neighbors), neural network models (e.g., feedforward, convolutional, recurrent, transformer-based), ensemble methods (e.g., bagging, boosting), or other classification techniques known to one skilled in the art. However, as with all hand-crafted radiomic efforts, it is equally valid to estimate PV statistics, or in fact the entirety of method 500 using any of the above classifiers directly with the image data.

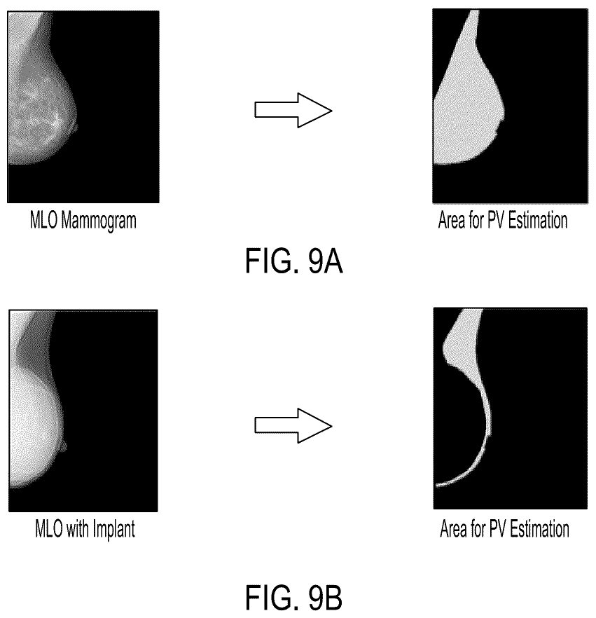

The PV statistics may be obtained for the parenchyma region, excluding highly attenuating features such as the pectoralis, implants and metal. FIGS. 9A-9B show example areas of MLO view mammograms considered for the PV statistics. PV statistics can also be obtained for other defined regions. The other defined regions can include, but are not limited to, the pectoralis, the interior of an implant, and/or the air region surrounding the breast.

Block 524 involves estimating the composition and shape of the target anatomical region (e.g., breast). Estimating the composition and shape of the target anatomical region (e.g., breast) may provide valuable insights into the patient's anatomy and help to optimize full-dose acquisition parameters. This estimation may involve, for example, an area estimation, a density estimation, an effective thickness estimation, a volume estimation, and/or a risk estimation. The area estimation may involve, given the detector element size, estimating the area of the target anatomical region (e.g., breast) using the detected anatomic landmarks. The method also allows the estimation of the composition and shape of individual features such as the nipple, parenchyma, pectoralis, metal, implant, etc. The density estimation may involve estimating the density of the target anatomical region (e.g., breast) using the detected anatomic landmarks. Anatomic landmarks in the breast can also include the detection of masses, calcifications, architectural distortion and other signs of malignant and benign breast disease detected in conjunction with machine learning algorithms, edge detection techniques, texture analysis techniques, and/or histogram-based techniques.

The effective thickness estimation may involve estimating the effective thickness of the target anatomical region (e.g., breast) using Beer-Lambert's law, the linear attenuation coefficients of adipose and glandular tissues, and the statistical information provided by the PV analysis in the air and/or in the parenchyma. The volume estimation may involve estimating the volume of the target anatomical region (e.g., breast) using the detected anatomic landmarks in conjunction with the estimated area and physical thickness, with the assistance of machine learning algorithms, edge detection techniques, texture analysis techniques, and/or histogram-based techniques. The risk estimation may involve estimating the present and future risk of cancer using area estimation, density estimation, effective thickness estimation, volume estimation, and/or additional measures of anatomic image features such as radiomic measures, machine learning algorithms, and/or other texture analysis methods.

Generally, these types of features fall under the description of morphologic features. The features listed above are an abbreviated list providing examples of appropriate morphologic features. Additional morphologic features could be used in this step. Such morphologic features include, but are not limited to, volume, surface area, surface to volume ratio, compactness, spherical disproportion, sphericity, asphericity, center of mass shift, maximum diameter, major axis length, minor axis length, least axis length, elongation, flatness, area density, convex hull, integrated intensity, Moran's I index, Geary's C measure, and/or others. Again, many of these features are considered radiomic features, and may be calculated using hand-crafted or AI methods.

Block 526 involves estimating the positioning of the target anatomical region (e.g., breast). This position estimation may provide insights about possible problems such as insufficient pectoralis coverage, nipple not in profile, inframammary fold missing, exaggerated CC, concave pectoralis, and/or cutoff. Generally, these measures of breast positioning vary by region and by professional organization. The measures listed above are an abbreviated list. Additional measures could be used in block 526.

In imaging the breast in mammography or tomosynthesis, it is important to image the pectoralis muscle as cancers may be located immediately adjacent or overlay the pectoralis. In a properly positioned mammogram, the pectoralis major muscle should be visible as a distinct border. Insufficient pectoralis coverage can result in missed lesions, particularly those located near the posterior aspect of the breast.

If the nipple is not positioned perpendicular to the long axis of the breast and parallel with the detector, this can lead to suboptimal visualization of the breast tissue. In a properly positioned mammogram, the nipple should be centered and pointing perpendicular to the chest-wall. If the nipple is not in profile, it may appear off-center or at an angle, leading to incomplete imaging of the surrounding breast tissue. Nipple positioning errors can result in missed lesions, particularly those located near the areola.

If the inframammary fold (IMF) is not visible or appears distorted, which can lead to incomplete visualization of the breast tissue. In a properly positioned mammogram, the IMF should be visible as a distinct border at the lower edge of the image. If the IMF is missing or appears distorted, it may indicate that the breast is not in optimal position. Missing IMF can result in missed lesions, particularly those located near the inferior aspect of the breast.

If the breast is compressed too tightly, this can lead to distortion and incomplete visualization of the breast tissue. In a properly positioned CC mammogram, the compression should be sufficient to flatten the breast against the imaging plate, but not so tight as to distort the tissue. If the CC is exaggerated, it may appear stretched or irregularly shaped, leading to incomplete imaging of the breast tissue. Exaggerated CC can result in missed lesions, particularly those located near the medial aspect of the breast.

If the pectoralis muscle appears concave, this can lead to incomplete visualization of the breast tissue. In a properly positioned mammogram, the pectoralis major muscle should appear as a flat or slightly convex (rounded) border at the edge of the image. If the pectoralis muscle is concave, it may indicate that the breast is not in optimal position. Concave pectoralis can result in missed lesions, particularly those located near the posterior aspect of the breast.

If the edge of the pre-shot image appears truncated or irregularly shaped then it is likely that the breast is “cut-off” meaning that the entire volume of the breast was not imaged. This can lead to incomplete visualization of the breast tissue. In a properly positioned mammogram, the edges of the image should appear smooth and rounded. If there is a cutoff, it may indicate that the breast was not aligned correctly within the field of view. Cutoff can result in missed lesions, particularly those located near the edge of the breast. Note that in instances of a large breast it may be necessary to image the breast in sections (tiles) that only cover part of the breast. In such instances, cutoff is a natural part of the imaging process. In such instances, the analysis of excessive tissue overlap between images, or missing tissue between images can be analyzed.

An image quality metric can be calculated for each of the above listed features related to breast positioning. Example methods of measuring image quality are described in U.S. Pat. No. 12,250,738.

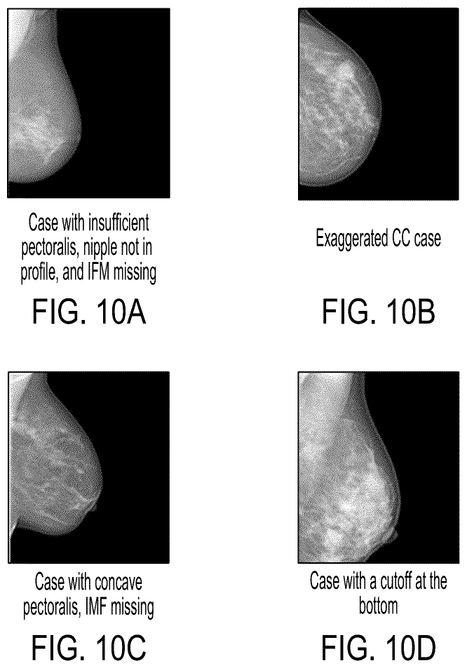

FIGS. 10A-10D illustrate some of the common breast positioning problems which can be detected using the present solution. FIG. 10A shows the case with insufficient pectoralis, nipple not in profile, and inframammary fold (IMF) missing. FIG. 10B shows the case of an exaggerated craniocaudal (CC) view. FIG. 10C shows the case with concave pectoralis and IMF missing. FIG. 10D shows the case of an image with a cutoff at the bottom.

After completing the operations of block 526, method 500 continues to block 528 where it ends or other operations are performed. The other operations can include, but are not limited to, returning to block 502 or going to block 414 of FIG. 4.

The present solution may be extended to other imaging modalities. Within the field of radiology, the present solution can be readily extended to provide insights into dose, composition, and positioning of other imaging modalities and organs through the analysis of the pre-shot image. The other imaging modalities can include, but are not limited to, tomosynthesis, chest x-ray imaging, musculoskeletal x-ray imaging (MSK), brain x-ray imaging, cardiovascular x-ray imaging, and/or dental x-ray imaging.

In the case of tomosynthesis, images of the patient are acquiring through a scanning motion of the x-ray source. During the scanning process, a series of projection images of an object are acquired at various scan angles relative to the object. In the context of tomosynthesis, a pre-shot image typically refers to a single low-dose projection acquired at or near the starting angle of scan; the full-dose image consists of the series of projection images that are acquired at different angles relative to the object (e.g., the breast or other body part). The series of projection images are then reconstructed into a stack of slices. The stack of slices may be a stack of slices parallel with the detector, or a multiplanar reconstruction (MPR) in which the slices are at an arbitrary angle to the detector. The reconstructed images may be of the whole breast or a region of interest. The region of interest may be the whole breast or a portion of the breast. The images can be reconstructed on a plane (for example as defined by three non-collinear points), or can be reconstructed on a non-planar surface. These images can be reconstructed as a full-dose radiograph or a synthetic 2D image. These images can be reconstructed as a volume from different viewpoints or a volume rendering. These images can also be used to reconstructed to create contrast-enhanced images.

In the chest x-ray imaging scenario, the present solution can be used to determine statistics of PV inside lungs, heart, bones, diaphragm and other tissues of interest. It can also be used to identify orthopedic hardware, pacemakers, radioactive seeds, among others. Insights about the patient's position can also be derived using the present solution. Some examples are incorrect rotation, inadequate inspiration, insufficient neck extension, heart shadows, and/or tilted clavicle.

In the MSK scenario, the present solution may be used to determine statistics of PV inside bones and soft tissue. It can also be used to identify orthopedic hardware, and other areas outside the tissues of interest. Insights about the patient's position can also be derived using the present solution. Some examples are insufficient bone coverage, incorrect joint alignment, inappropriate bone rotation, insufficient flexion of the knee (in hip imaging), insufficient extension of the elbow (in shoulder imaging), and/or insufficient extension of the cervical or lumbar spine (in spine imaging).

In the brain x-ray imaging scenario, the present solution can be used to determine statistics of PV inside different brain and skull structures while excluding objects that are not of interest such as a surgical halo. Insights about the patient's position can also be derived using the present solution. For example, the present solution may be used to obtain a scan asymmetry, an alignment of the Frankfort plane, and/or an inadequate extension of the neck and/or head.

In the cardiovascular x-ray imaging scenario, the present solution may be used to determine statistics of PV inside different regions of interest (such as the heart, great vessels and/or coronary arteries), while excluding other objects (such as pacemakers, large calcified lesions, artificial valves, stents, and/or catheters).

In the dental x-ray imaging scenario, the present solution may be used to determine statistics of PV within different regions (such as the teeth, periodontal structures, jawbone and/or facial bone structures). The present solution may be used to detect and remove special objects from the statistics (such as metal hardware present in the jaw or teeth). The present solution may also be used to detect patient positioning problems (such as insufficient or incorrect horizontal/vertical angulation of the patient head, and/or insufficient bite opening).

The present solution is applicable to conventional 2D x-ray imaging, but it can also be extended to fluoroscopy, angiography, computed tomography, cone-beam computed tomography, tomosynthesis, dual-energy and multi-energy imaging, photon counting imaging, and/or among others. This includes digital breast tomosynthesis (DBT), breast computed tomography, breast cone-beam computed tomography, contrast enhanced mammography, and/or contrast-enhanced tomosynthesis. The present solution may also be readily applied to other non-ionizing medical imaging modalities. The other non-ionizing medical imaging modalities can include, but are not limited to, magnetic resonance imaging (MRI), and/or ultrasound.

In the MRI scenario, the present solution can provide insights for better definition of acquisition parameters through the pre-shot images, such as the localizer pre-shot, the sagittal and coronal localizer pre-shots. Instead of saving radiation dose (since this is a non-ionizing imaging modality), the proper definition of acquisition parameters may help reduce scan time and improve image quality. The MRI acquisition parameters can include, but are not limited to, repetition time (TR), echo time (TE), flip angle, inversion time (TI), slice thickness, field of view (FOV), and/or resolution. The present solution may provide information about special objects and patient positioning similarly to those in x-ray imaging modalities listed above.

In ultrasound imaging the radiologist may opt to acquire pre-shot images (e.g., using B-mode scans) to define the location and depth of structures of interest for subsequent procedures such as ultrasound-guided injections or biopsy. In those cases, the present solution can be used to help define acquisition parameters and locate areas of interest, as well as detect problems with probe positioning. Additional ultrasound methods (such as A-mode, M-mode, doppler, color doppler, power doppler, and/or compounding) could be aided by the present solution.

The present solution provides a transformative approach to medical imaging by unlocking the potential of pre-shot images to enhance the image acquisition process. By systematically analyzing the information contained within these preliminary pre-shot images, the present solution not only facilitates accurate estimation of the full-dose image acquisition parameters but also helps ensure that the final imaging output is of relatively high quality. The choice of acquisition parameters vary depending on the modality. The factors considered in the analysis can include, but are not limited to, positioning, compression, exposure, contrast, sharpness, noise, artifacts, and/or labeling. The present solution provides a significant innovation in the landscape of medical imaging, contributing positively to clinical outcomes and patient care.

In general, the images acquired by the detector are called “for-processing images” prior to processing. This terminology can apply to the pre-shot or the full-dose images, but is most commonly used to describe the full-dose image. In general, the final images produced by the image processing or image reconstruction software are called “for-presentation” images, as these images are generally intended for presentation to the radiologist for review. However, it should be noted that these for-presentation images can be processed for presentation to a computer aided design (CAD) software, image triage software, radiomic or other quantitative assessment software, etc. It is not uncommon that the for-presentation images can differ based upon the intended target (radiologist, CAD, etc.).

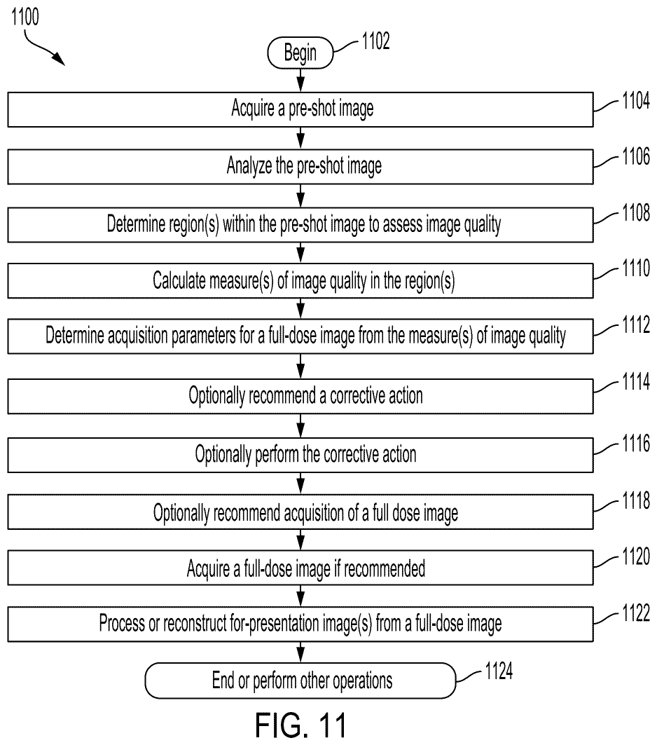

FIG. 11 is another illustration of a method 1100 for analyzing and using a pre-shot image to determine the acquisition parameters for a full-dose image. Method 1100 begins at block 1102. In block 1104, the pre-shot image is acquired by an imaging device (e.g., imaging device 110 of FIG. 1). The imaging device can include, but is not limited to, a full-field digital mammography (FFDM) system, a 3D tomosynthesis imaging system, a biopsy imaging system, a contrast-enhanced mammography (CEM) system, a magnetic resonance imaging (MRI) system, or an ultrasound system.

The pre-shot image is analyzed in block 1106 by a computing device (e.g., computing device 102 of FIG. 1) and/or a processor (e.g., processor 302 of FIG. 3). This analysis can involve the operations of blocks 1108-1118. Blocks 1108-1118 involve: (1108) determining one or more regions within the image to assess image quality; (1110) calculating one or more measures of image quality in the one or more regions; (1112) determining the acquisition parameters for a full-dose image from the one or more measures of image quality; (1114) recommending a corrective action; (1116) performing the corrective action; and/or (1118) recommending acquisition of a full-dose image.

The full-dose image is acquired by the imaging device in block 1120. In block 1122, one or more for-presentation images are reconstructed from a full-dose image. The full-dose image may be prepared for presentation by, for example: creating a copy of the full-dose image; perform an iterative reconstruction process to improve the image quality and reduce noise; suppressing noise and/or artifacts; adjusting the brightness and/or contrast of the image to highlight specific structures, tissues or objects; overlying masks on the image highlight specific structures, tissues or objects; cropping the image; and/or resizing the image rotating the image.

Subsequently, method 1100 continues to block 1124 where it ends or other operations are performed. The other operations can include, but are not limited to, returning to block 1102, presenting the for-presentation image on a display screen (e.g., display unit 312 of FIG. 3), or going to block 424 of FIG. 4.

As noted above, example methods of measuring image quality are described in U.S. Pat. No. 12,250,738, the disclosure of which is fully incorporated into this document by reference. Those methods include generating an implant mask for an implant region of the digital image in which an implant appears. The system also may generate masks for other features, such as collimator blades, compression paddle arms, spot paddles, surgical clips or markers placed on the target anatomical region during imaging. The individual 2D masks may be processed to generate a combined mask, which may be a 3D mask.

Once the system has generated a combined mask as described above, the system may use the individual masks and/or the combined mask to generate a measure of quality of the image. One way by which the system may assess quality is to assess whether the breast was properly oriented in the combined mask image. For examine, where the image is a mammogram, the system may draw a line from the nipple mask to a nearest border point on a border of the pectoralis mask. The system may then measure an angle formed by the intersection of the line with the line representing the border. The system may then generate and report a measure of quality of the image based on the measured angle. In particular, the system may determine that the image is of satisfactory quality if the angle is 90 degrees or approximately 90 degrees; otherwise, the system may determine that the image is not of satisfactory quality. In this context, the term “approximately” may include any range up to +/−25%, such that “approximately 90 degrees” includes ranges including 67.5 to 112.5 degrees (+/−25%), along with any subrange within that range, such as but not limited to 81 to 99 degrees (+/−10%), 76.5 to 103.5 degrees (+/−15%), and 72 to 108 degrees (+/−20%).

The quality assessment also may consider the individual probability masks, as noted above. For example, if no mask of a particular region can be generated, or if the probability of a mask being associated with a particular region is below a threshold, then the system may determine that the image quality is below the threshold.

If the image quality measure is above the threshold, then the system may classify the image as of acceptable quality. If the image quality measure is below the threshold, then the system may classify the image is of unacceptable quality.

In view of the above discussion, the present solution concerns implementing systems and methods for analyzing images. The methods include those of any of the following clauses:

Clause 1: A method comprising: (a) acquiring, by an imaging device, a pre-shot image of a target anatomical area of an individual; (b) analyzing, by a processor, the pre-shot image to calculate one or more measures of image quality in one or more regions within the pre-shot image, and determine acquisition parameters for a full-dose image from the one or more measures of image quality; (c) acquiring, by the imaging device, a full-dose image using the acquisition parameters determined during analysis of the pre-shot image; and (d) generating a final image from the full-dose image.

Clause 2: The method according to clause 1, wherein the analyzing comprises detecting one or more anatomic landmarks in the pre-shot image.

Clause 3: The method according to clause 2, wherein the one or more anatomic landmarks comprise one or more of a nipple, a pectoralis muscle, and a parenchyma.

Clause 4: The method according to clause 2, wherein the analyzing further comprises estimating pixel value statistics for the one or more anatomic landmarks.

Clause 5: The method according to any of clauses 1-4, wherein the analyzing comprises detecting one or more occlusions in the pre-shot image.

Clause 6: The method according to clause 5, wherein the one or more occlusions comprise one or more of an implant, a pacemaker, metal, spot paddles, or biopsy apparatus.

Clause 7: The method according to any of clauses 1-6, wherein the analyzing comprises detecting one or more image testing objects in the pre-shot image, wherein the one or more image testing objects comprise a phantom.

Clause 8: The method according to clause 7, wherein the analyzing further comprises estimating pixel value statistics for the one or more image testing objects.

Clause 9: The method according to any of clauses 1-8, wherein the analyzing comprises estimating a scatter map for objects in the pre-shot image.

Clause 10: The method according to any of clauses 1-9, wherein the analyzing comprises estimating a composition and shape of the target anatomical area.

Clause 11: The method according to any of clauses 1-10, wherein the analyzing comprises estimating a positioning of the target anatomical area relative to the imaging device.

Clause 12: The method according to any of clauses 1-11, wherein determining the acquisition parameters comprises (i) obtaining a pre-defined value for an acquisition parameter that is to be used for the full-dose image, or (ii) selecting a value for acquisition parameters that is different than a pre-defined value based on information provided by analyzing the pre-shot image.

Clause 13: A system, comprising a processor and a non-transitory computer-readable medium that stores instructions that are configured to, when executed by the processor, cause the processor to: (a) obtain a pre-shot image of a target anatomical area of an individual that was acquired by an imaging device; (b) analyze the pre-shot image to calculate one or more measures of image quality in one or more regions within the pre-shot image, and determine acquisition parameters for a full-dose image from the one or more measures of image quality; (c) control an imaging device to acquire a full-dose image using the acquisition parameters determined during analysis of the pre-shot image; and (d) generate a final image from the full-dose image.

Clause 14: The system according to clause 13, wherein the instructions are further configured to cause the processor to detect one or more anatomic landmarks in the pre-shot image.

Clause 15: The system according to clause 14, wherein the one or more anatomic landmarks comprise one or more of a nipple, a pectoralis muscle, and a parenchyma.

Clause 16: The system according to clause 14, wherein the instructions are further configured to cause the processor to estimate pixel value statistics for the one or more anatomic landmarks.

Clause 17: The system according to any of clauses 13-16, wherein the instructions are further configured to cause the processor to detect one or more occlusions in the pre-shot image.

Clause 18: The system according to clause 17, wherein the one or more occlusions comprise one or more of an implant, a pacemaker, metal, spot paddles, or biopsy apparatus.

Clause 19: The system according to any of clauses 13-18, wherein the instructions are further configured to cause the processor to detect one or more image testing objects in the pre-shot image, wherein the one or more image testing objects comprise a phantom.

Clause 20: The system according to any of clauses 13-19, wherein the instructions are further configured to cause the processor to estimate one or more of the following: (i) a scatter map for one or more objects in the pre-shot image; (ii) pixel value statistics for the p one or more objects in the pre-shot image; (iii) a composition and shape of the target anatomical area; or (iv) a positioning of the target anatomical area relative to the imaging device.

Clause 21: The system according to any of clauses 13-20, wherein the instructions to determine the acquisition parameters comprise instructions to (i) obtain a pre-defined value for an acquisition parameter that is to be used for the full-dose image or (ii) dynamically select a value for acquisition parameters that is different than the pre-defined value based on information provided by analyzing the pre-shot image.

Clause 22: The system according to any of clauses 13-21, wherein the imaging device comprises a full-field digital mammography system, a 3D tomosynthesis imaging system, a biopsy imaging system, a contrast-enhanced mammography system, a magnetic resonance imaging system, or an ultrasound system.

Reference throughout this specification to “one embodiment”, “an embodiment”, or similar language means that a particular feature, structure, or characteristic described in connection with the indicated embodiment is included in at least one embodiment of the present solution. Thus, the phrases “in one embodiment”, “in an embodiment”, and similar language throughout this specification may, but do not necessarily, all refer to the same embodiment.

As used in this document, the singular form “a”, “an”, and “the” include plural references unless the context clearly dictates otherwise. Unless defined otherwise, all technical and scientific terms used herein have the same meanings as commonly understood by one of ordinary skill in the art. As used in this document, the term “comprising” means “including, but not limited to”.

In this document, when terms such as “first” and “second” are used to modify a noun, such use is simply intended to distinguish one item from another, and is not intended to require a sequential order unless specifically stated.

The described features, advantages and characteristics disclosed herein may be combined in any suitable manner. One skilled in the relevant art will recognize, in light of the description herein, that the disclosed systems and/or methods can be practiced without one or more of the specific features. In other instances, additional features and advantages may be recognized in certain scenarios that may not be present in all instances.

Although the systems and methods have been illustrated and described with respect to one or more implementations, equivalent alterations and modifications will occur to others skilled in the art upon the reading and understanding of this specification and the annexed drawings. In addition, while a particular feature may have been disclosed with respect to only one of several implementations, such feature may be combined with one or more other features of the other implementations as may be desired and advantageous for any given or particular application. Thus, the breadth and scope of the disclosure herein should not be limited by any of the above descriptions. Rather, the scope of the invention should be defined in accordance with the following claims and their equivalents.

1. A method for analyzing images, comprising:
acquiring, by an imaging device, a pre-shot image of a target anatomical area of an individual;
analyzing, by a processor, the pre-shot image to:
calculate one or more measures of image quality in one or more regions within the pre-shot image; and
determine acquisition parameters for a full-dose image from the one or more measures of image quality;
acquiring, by the imaging device, a full-dose image using the acquisition parameters determined during analysis of the pre-shot image; and
generating a final image from the full-dose image.

2. The method according to claim 1, wherein the analyzing comprises detecting one or more anatomic landmarks in the pre-shot image.

3. The method according to claim 2, wherein the one or more anatomic landmarks comprise one or more of a nipple, a pectoralis muscle, and a parenchyma.

4. The method according to claim 2, wherein the analyzing further comprises estimating pixel value statistics for the one or more anatomic landmarks.

5. The method according to claim 1, wherein the analyzing comprises detecting one or more occlusions in the pre-shot image.

6. The method according to claim 5, wherein the one or more occlusions comprise one or more of an implant, a pacemaker, metal, spot paddles, or biopsy apparatus.

7. The method according to claim 1, wherein the analyzing comprises detecting one or more image testing objects in the pre-shot image, wherein the one or more image testing objects comprise a phantom.

8. The method according to claim 7, wherein the analyzing further comprises estimating pixel value statistics for the one or more image testing objects.

9. The method according to claim 1, wherein the analyzing comprises estimating a scatter map for objects in the pre-shot image.

10. The method according to claim 1, wherein the analyzing comprises estimating a composition and shape of the target anatomical area.

11. The method according to claim 1, wherein the analyzing comprises estimating a positioning of the target anatomical area relative to the imaging device.

12. The method according to claim 1, wherein determining the acquisition parameters comprises (i) obtaining a pre-defined value for an acquisition parameter that is to be used for the full-dose image, or (ii) dynamically selecting a value for acquisition parameters that is different than the pre-defined value based on information provided by analyzing the pre-shot image.

13. A system, comprising:
a processor; and
a non-transitory computer-readable medium that stores instructions that are configured to, when executed by the processor, cause the processor to:
obtain a pre-shot image of a target anatomical area of an individual that was acquired by an imaging device,
analyze the pre-shot image to:
calculate one or more measures of image quality in one or more regions within the pre-shot image; and
determine acquisition parameters for a full-dose image from the one or more measures of image quality,
control an imaging device to acquire a full-dose image using the acquisition parameters determined during analysis of the pre-shot image, and
generate a final image from the full-dose image.

14. The system according to claim 13, wherein the instructions are further configured to cause the processor to detect one or more anatomic landmarks in the pre-shot image.

15. The system according to claim 14, wherein the one or more anatomic landmarks comprise one or more of a nipple, a pectoralis muscle, and a parenchyma.

16. The system according to claim 14, wherein the instructions are further configured to cause the processor to estimate pixel value statistics for the one or more anatomic landmarks.

17. The system according to claim 13, wherein the instructions are further configured to cause the processor to detect one or more occlusions in the pre-shot image.

18. The system according to claim 17, wherein the one or more occlusions comprise one or more of an implant, a pacemaker, metal, spot paddles, or biopsy apparatus.

19. The system according to claim 13, wherein the instructions are further configured to cause the processor to detect one or more image testing objects in the pre-shot image, wherein the one or more image testing objects comprise a phantom.

20. The system according to claim 13, wherein the instructions are further configured to cause the processor to estimate one or more of the following:
pixel value statistics for one or more objects in the pre-shot image;
a scatter map for the objects in the pre-shot image;
a composition and shape of the target anatomical area; or
a positioning of the target anatomical area relative to the imaging device.

21. The system according to claim 13, wherein the instructions to determine the acquisition parameters comprise instructions to (i) obtain a pre-defined value for an acquisition parameter that is to be used for the full-dose image, or (ii) dynamically select a value for acquisition parameters that is different than the pre-defined value based on information provided by analyzing the pre-shot image.

22. The system according to claim 13, wherein the imaging device comprises a full-field digital mammography system, a 3D tomosynthesis imaging system, a biopsy imaging system, a contrast-enhanced mammography system, a magnetic resonance imaging system, or an ultrasound system.
